(19) United States
(12) Patent Application Publication
(10) Pub. No.: US 2019/0271603 A1
(43) Pub. Date: Sep. 5, 2019

(54) WOVEN ARTICLE AND METHOD FOR DETECTING STRESS DISTRIBUTION OF WOVEN ARTICLE UNDER STRESS

(71) Applicant: SOUTHERN TAIWAN UNIVERSITY OF SCIENCE AND TECHNOLOGY, Tainan City (TW)

(72) Inventors: YI-CHUN DU, TAINAN CITY (TW); CHENG-PANG SHIH, TAINAN CITY (TW); JING-YI CHEN, TAINAN CITY (TW)

(21) Appl. No.: 15/911,405

(22) Filed: Mar. 5, 2018

(51) Int. Cl. G01L 5/16; D03D 15/00; D03D 9/00; G01L 1/20; A61B 5/00

(52) U.S. Cl. CPC G01L 5/161; D03D 15/00; D03D 9/00; G01L 1/205; D10B 2501/00; A61B 5/6804; D03D 2700/0166; D10B 2509/00; A61B 5/4519

(57) ABSTRACT

A woven article and a method for detecting a stress distribution of the woven article under stress are provided. The woven article includes a woven body and a plurality of metallic yarns connected to the woven body and spaced apart from each other. The woven body defines more than one direction thereon. The metallic yarns each extend in the direction. A resistance sensor is connected with each of the metallic yarns. A stress is exerted on the woven body, and the resistance sensor detects a resistance value generated by the metallic yarns on the woven body. According to the resistance value detected by the resistance sensor, a stress distribution of the woven body under stress can be obtained.

### FIELD OF THE INVENTION

The present invention relates to a woven article and a method for detecting a stress distribution of the woven article under stress, and more particularly, to a method to obtain the stress distribution through the change of the resistance value after a woven article is under stress.

### BACKGROUND OF THE INVENTION

Long sitting, long standing, bad posture or excessive exercise may cause varicose veins in the legs or neck/waist soreness, and the muscle stress will change accordingly.

That is, by obtaining abnormal changes of muscle stress, people can instantly correct the bad posture or take a rest.

A conventional method to know the change of muscle stress uses many stress detectors. The stress detectors are attached to the muscles to get the change of muscle stress.

However, the stress detectors are expensive. The method of using many stress detectors to know the stress on the muscles is not cost-effective.

### SUMMARY OF THE INVENTION

The primary object of the present invention is to provide a woven article and a method for detecting a stress distribution of the woven article under stress.

According to one aspect of the present invention, a method for detecting a stress distribution of a woven article under stress is provided. The method comprises the steps of: defining more than one direction on a woven body; connecting a plurality of spaced metallic yarns to the woven body, the metallic yarns each extending in the direction; exerting a stress on the woven body and providing a resistance sensor to detect a resistance value generated by the metallic yarns on the woven body; and obtaining a stress distribution of the woven body under stress according to the resistance value detected by the resistance sensor.

Preferably, the metallic yarns are combined with the woven body in a plurality of directions so that the metallic yarns are arranged in a staggered manner. The metallic yarns generating the resistance value in different directions are intersected to form a compressive area. In different compressive areas, a stress distribution ratio of each of the compressive areas is obtained by averaging the resistance values of the plurality of metal yarns of each of the compressive areas. After an actual stress of one of the compressive areas is measured, the actual stress of each of the compressive areas is obtained according to the stress distribution ratio.

Preferably, the metallic yarns are combined with the woven body in three to five directions so that the metallic yarns are staggered in the three to five directions to obtain the compressive area.

According to another aspect of the present invention, a woven article is provided. The woven article comprises a woven body, a plurality of metallic yarns, and a resistance sensor. The woven body defines more than one direction thereon. The metallic yarns are connected to the woven body and spaced apart from each other. The metallic yarns each extend in the direction. The resistance sensor is connected with each of the metallic yarns.

Preferably, a distance between adjacent two of the metallic yarns in the same direction is in the range of 2-4 cm.

Preferably, the distance between adjacent two of the metallic yarns in the same direction is 3 cm.

Preferably, the metallic yarns are combined with the woven body in three to five directions so that the metallic yarns are staggered in the three to five directions.

Preferably, the resistance sensor is connected with each of the metallic yarns in a vertical direction thereof.

Preferably, each of the metallic yarns is connected to the woven body in a criss-cross pattern.

According to the above technical features, the present invention can achieve the following effects:

1. When the woven article is under stress, through the change of the resistance value, the stress distribution can be obtained. When there are multiple compressive areas, the resistance ratio between the multiple compressive areas can be obtained. By detecting the actual stress of a single compressive area, the actual stress of the other multiple compressive areas can be obtained according to the resistance ratio.

2. The metallic yarns are cheap. After multiple compressive areas are detected, only a single stress detector is used to detect the actual stress of a single compressive area to obtain the actual stress of all the compressive areas. Thus, the cost of use is lower.

### BRIEF DESCRIPTION OF THE DRAWINGS

FIG. 1 is a perspective view of the compression stocking as the woven article of the present invention;

FIG. 2 is an exploded view of the woven article of the present invention, showing that the woven body is combined with the metallic yarns in different directions;

FIG. 3 is a planar enlarged view of the area A of FIG. 1;

FIG. 4 is a schematic view of the present invention, showing that each metallic yarn is connected to the woven body in a criss-cross pattern;

FIG. 5 is a schematic view of the present invention when in use, showing that the compression stocking is worn on the user's shank;

FIG. 6 is a planar enlarged view of the area B of FIG. 5;

FIG. 7 is a schematic view of another embodiment of the woven article of the present invention; and

FIG. 8 is a schematic view of the compression garment as the woven article of the present invention.

### DETAILED DESCRIPTION OF THE PREFERRED EMBODIMENTS

Embodiments of the present invention will now be described, by way of example only, with reference to the accompanying drawings.

Based on the above technical features, the effects of the woven article and the method for detecting the stress distribution of the woven article under stress of the present invention will be clearly shown in the following embodiments.

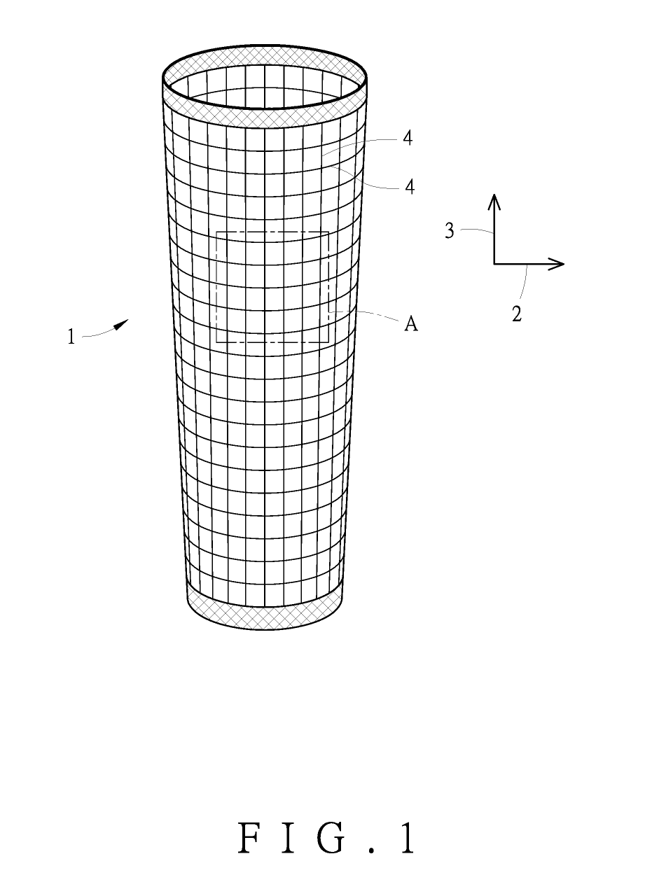

Referring to FIG. 1, a medical compression stocking is taken as an example for the woven article of the present invention and comprises a woven body (1).

The woven body (1) defines more than one direction thereon, including a first direction (2) and a second direction (3) in this embodiment. A plurality of metallic yarns (4) are provided and connected on the woven body (1).

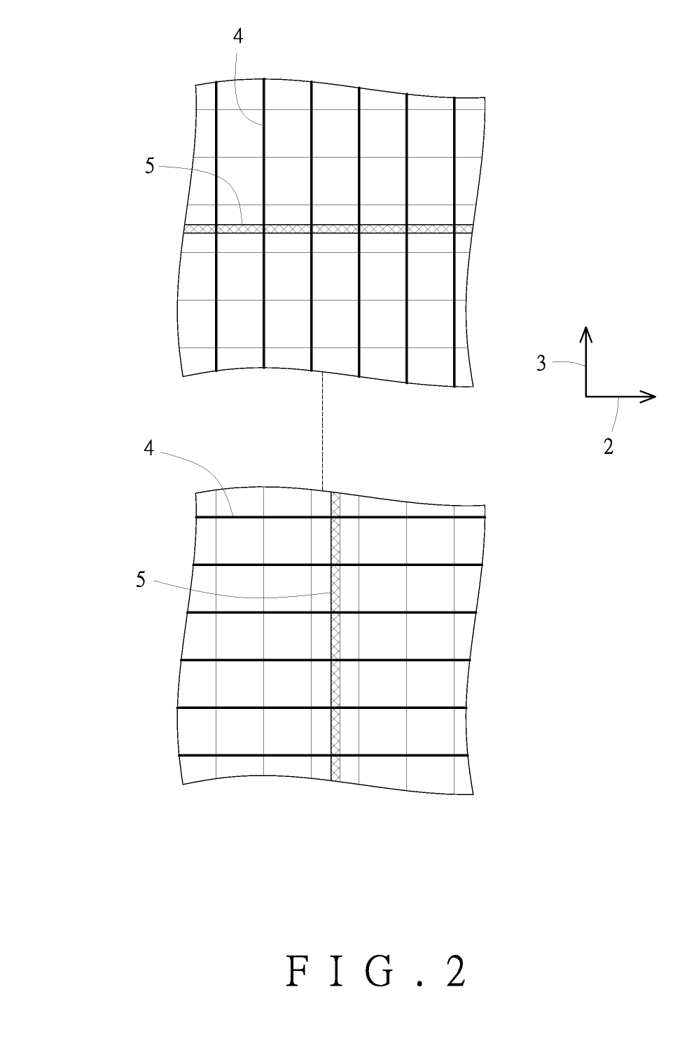

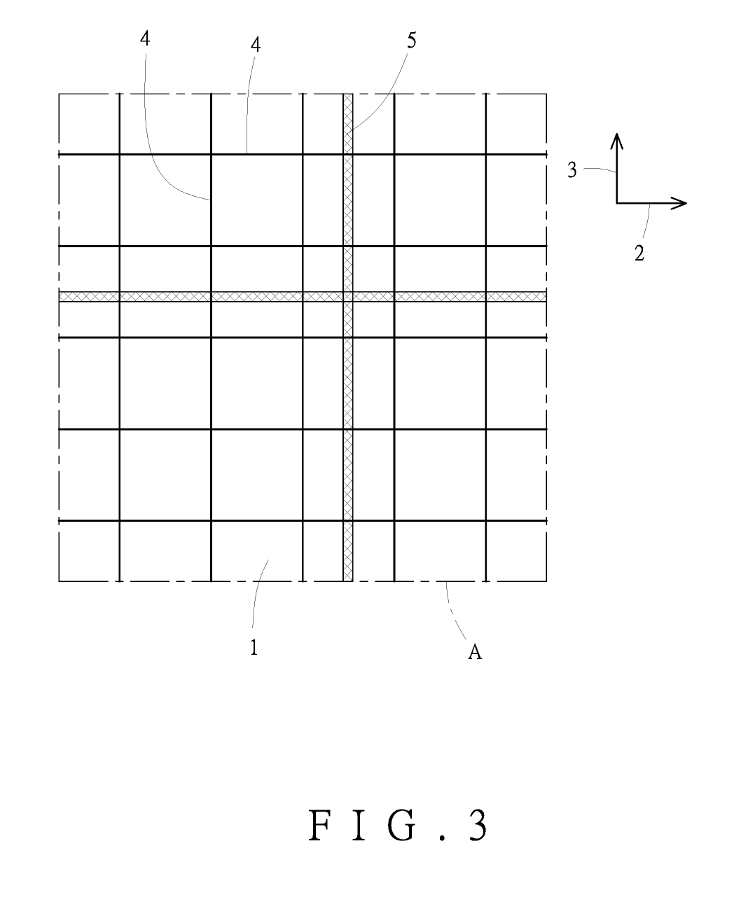

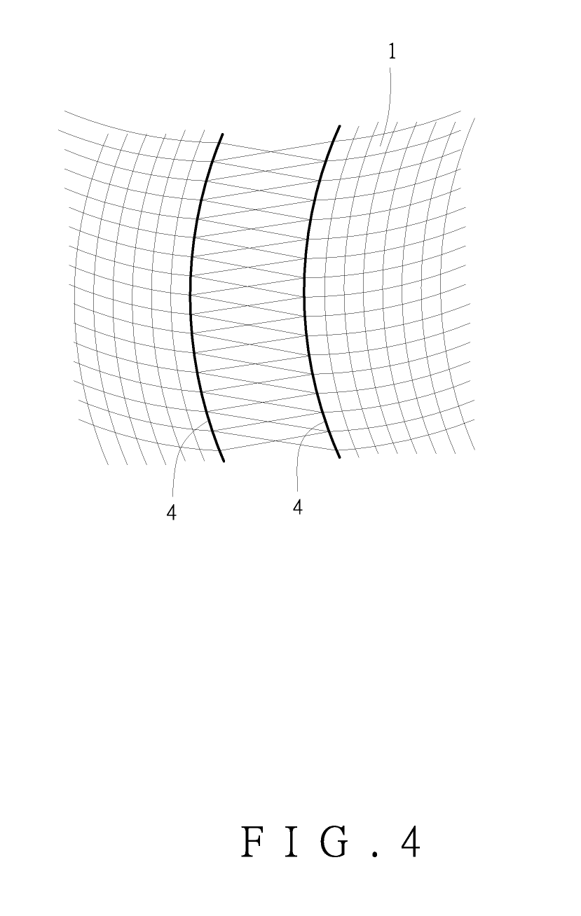

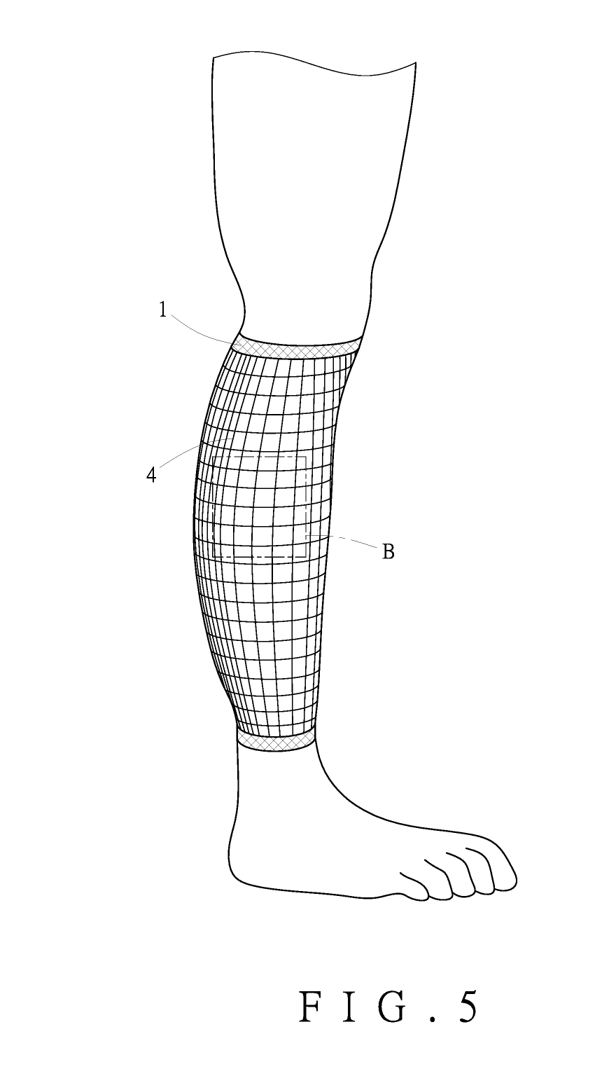

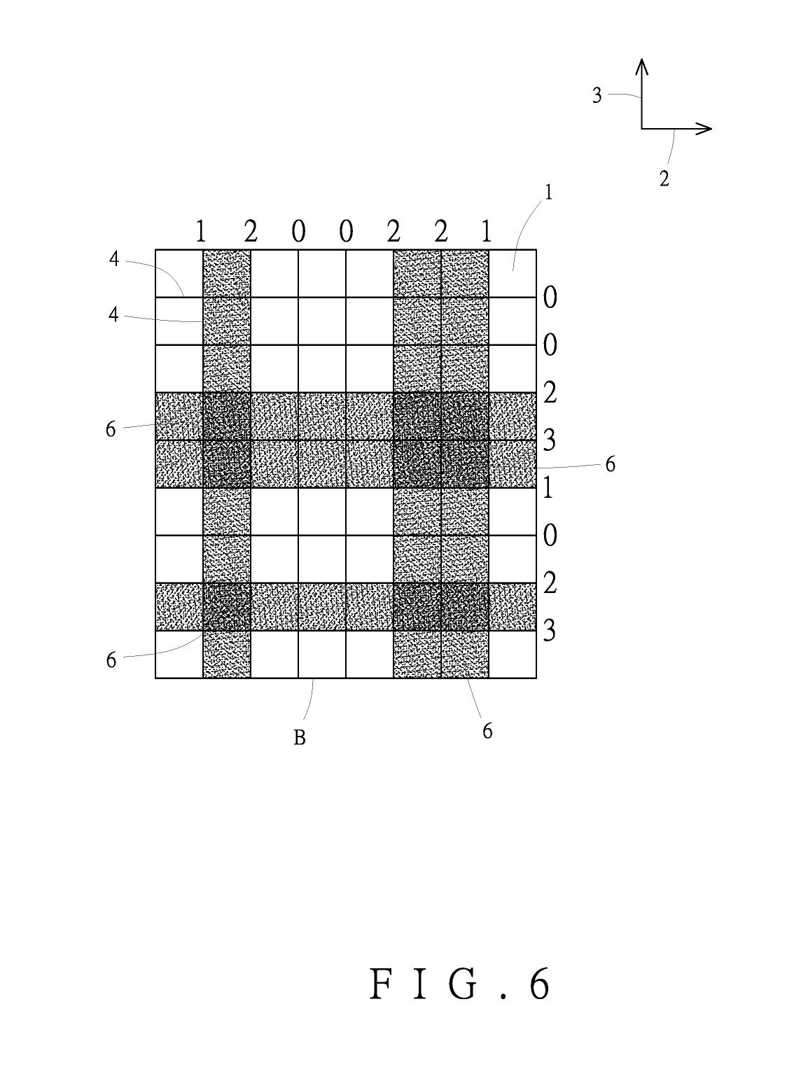

Referring to FIG. 2 to FIG. 4, the metallic yarns (4) in each direction are stacked in a layered manner such that the metallic yarns (4) are arranged on the woven body (1) in a staggered manner along the first direction (2) and the second direction (3). The distance between adjacent two of the metallic yarns (4) in the same direction is in the range of 2-4 cm. In this embodiment, the distance between adjacent two of the metallic yarns (4) in the same direction is 3 cm. Each metallic yarn (4) is connected to the woven body (1) in a criss-cross pattern. A resistance sensor (5) is connected with each of the metallic yarns (4). In this embodiment, the resistance sensor (5) is connected with each of the metallic yarns (4) in the vertical direction thereof. Referring to FIG. 5 and FIG. 6, the woven body (1) is worn on an object to be measured. In this embodiment, the medical compression stocking is worn on the user's shank. When the user does an exercise with stress on each part of the shank, the corresponding woven body (1) and the metallic yarns (4) on the woven body (1) are deformed under stress. At this time, the resistance sensor (5) (as to the resistance sensor (5), please see FIG. 3), on the metal spinning yarns (4) will detect a resistance value generated by the deformation of each of the metal spinning yarns (4).

Therefore, according to the resistance values generated by the metallic yarns (4), the stress distribution of the user's shank under stress can be obtained. As shown in FIG. 6, the metallic yarns (4) generating the resistance value in the first direction (2) and the second direction (3) are intersected to form a compressive area (6). In the figures, there are four compressive areas (6) due to the intersection. In different compressive areas (6), the stress distribution ratio of each of the compressive areas (6) can be obtained by averaging the resistance values of the plurality of metal yarns (4) in each of the different compressive areas (6).

The user uses a stress detector to measure the actual stress of one of the compressive areas (6) (using the stress detector to detect the stress on the user's shank is the prior art, so it won't be described hereinafter). The actual stress of each compressive area (6) can be obtained according to the stress distribution ratio of each compressive area (6). The metallic yarn (4) is cheaper than the stress detector. Therefore, after multiple compressive areas (6) are detected, only a single stress detector is used to detect the actual stress of a single compressive area (6) to obtain the actual stress of all the compressive areas (6). Thus, the cost of use is lower.

Furthermore, the distance between two adjacent metallic yarns (4) is 3 cm. This spacing design can detect the different compressive areas (6) on the user's shank more accurately.

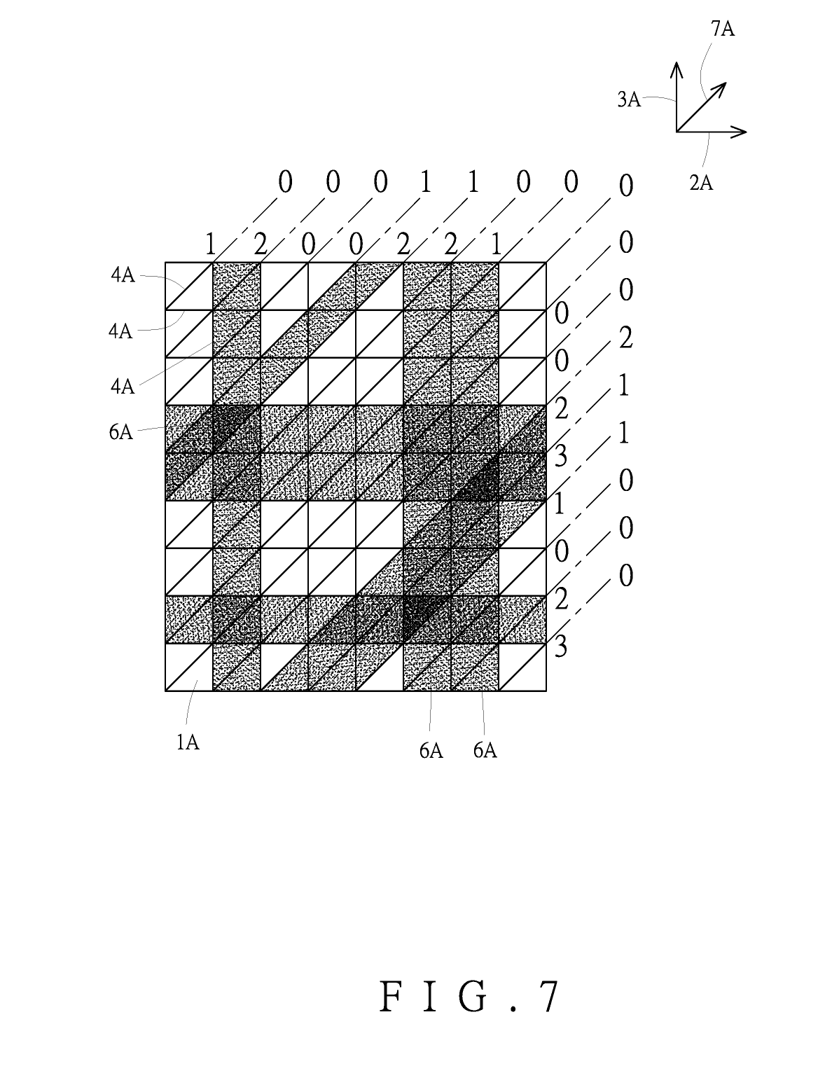

Referring to FIG. 7, a plurality of metallic yarns (4A) may be combined with a woven body (1A) in three to five directions. As shown in FIG. 7, the metallic yarns (4A) are staggered in a first direction (2A), a second direction (3A) and a third direction (7A) so as to obtain a more accurate compressive area (6A). The combination of the metallic yarns in more directions can detect the compressive area more accurately. However, the metallic yarns arranged in layers in too many directions will make the final woven article too thick, so the metallic yarns combined in three to five directions will be a better design.

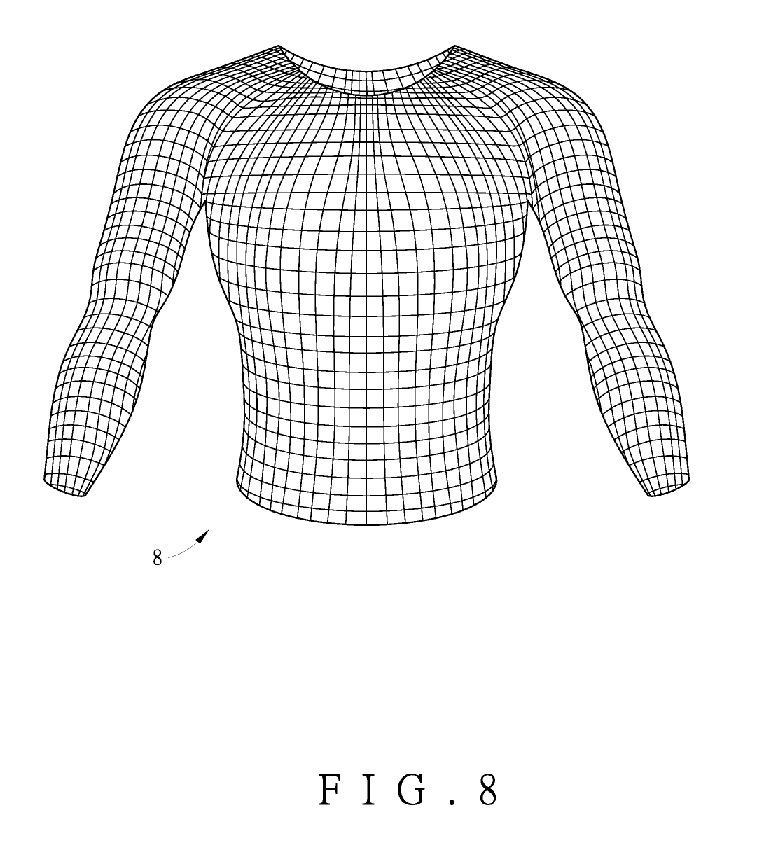

Referring to FIG. 8, in addition to the compression stocking, the woven article of the present invention may be made into a compression garment (8) for burns or other similar wearing apparel.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A method for detecting a stress distribution of a woven article under stress, comprising the steps of:
defining more than one direction on a woven body;
connecting a plurality of spaced metallic yarns to the woven body, the metallic yarns each extending in the direction;
exerting a stress on the woven body and providing a resistance sensor to detect a resistance value generated by the metallic yarns on the woven body; and
obtaining a stress distribution of the woven body under stress according to the resistance value detected by the resistance sensor.

2. The method as claimed in claim 1, wherein the metallic yarns are combined with the woven body in a plurality of directions so that the metallic yarns are arranged in a staggered manner; the metallic yarns generating the resistance value in different directions are intersected to form a compressive area, in different compressive areas, a stress distribution ratio of each of the compressive areas is obtained by averaging the resistance values of the plurality of metal yarns of each of the compressive areas; after an actual stress of one of the compressive areas is measured, the actual stress of each of the compressive areas is obtained according to the stress distribution ratio.

3. The method as claimed in claim 2, wherein the metallic yarns are combined with the woven body in three to five directions so that the metallic yarns are staggered in the three to five directions to obtain the compressive area.

4. A woven article, comprising:
a woven body, the woven body defining more than one direction thereon;
a plurality of metallic yarns, the plurality of metallic yarns being connected to the woven body and spaced apart from each other, the metallic yarns each extending in the direction; and
a resistance sensor, connected with each of the metallic yarns.

5. The woven article as claimed in claim 4, wherein a distance between adjacent two of the metallic yarns in the same direction is in the range of 2-4 cm.

6. The woven article as claimed in claim 5, wherein the distance between adjacent two of the metallic yarns in the same direction is 3 cm.

7. The woven article as claimed in claim 4, wherein the metallic yarns are combined with the woven body in three to five directions so that the metallic yarns are staggered in the three to five directions.

8. The woven article as claimed in claim 4, wherein the resistance sensor is connected with each of the metallic yarns in a vertical direction thereof.

9. The woven article as claimed in claim 4, wherein each of the metallic yarns is connected to the woven body in a criss-cross pattern.
